(19) United States
(12) Patent Application Publication
(10) Pub. No.: US 2009/0234305 A1
(43) Pub. Date: Sep. 17, 2009

(54) TEMPORARY SKIN SUBSTITUTE COMPRISED OF BIOLOGICAL COMPOUNDS OF PLANT AND ANIMAL ORIGINS

(76) Inventor: Ernest Aubrey Woodroof, Calsbad, CA (US)

Correspondence Address: STEVEN W. WEBB, 825 College Blvd., Suite 102620, Oceanside, CA, 92057, US

(21) Appl. No.: 12/348,412

(22) Filed: Jan. 5, 2009

Related U.S. Application Data
(63) Continuation-in-part of application No. 12/049,321, filed on Mar. 15, 2008.

(51) Int. Cl. A61F 13/00

(52) U.S. Cl. 604/290; 604/304

(57) ABSTRACT

An improved skin substitute is presented comprised of non-biological materials produced with a series of regularly-spaced pores and a nylon mesh netting. The top component is a thin (approximately 0.001″ thick) silicone elastomer in which pore holes have been vacuum-pulled; physically attached to the silicone elastomer is a fine knitted nylon fabric (12/1, 15/2 denier); incorporated into the silicone/nylon structure are several wound healing agents, including Vitamin E, Vitamin C, Chondroitin 4 Sulfate, Chondroitin 6 Sulfate and Immuno-10 (a highly purified fraction of Aloe Vera) in addition to porcine collagen peptide.

The holes in the skin substitute provide a range of porosities to ensure minimum fluid accumulation beneath the skin substitute without wound desiccation. The three-dimensional nature of the nylon mesh in addition to the use of collagen peptides provides better acute adherence with a structure that supports the growth of new skin cells. The range of hole diameters preferred in the present invention is 0.75 mm to 1.05 mm and at holes centered at ¼″-⅓″, providing minimal fluid accumulation beneath the Temporary Skin Substitute, which will reduce infection complications and maximize wound healing.

### RELATED APPLICATIONS

This application is a Continuation-in-Part of application Ser. No. 12/049,321 which is currently copending.

### FIELD OF THE INVENTION

This invention relates to the fields of artificial skin substitutes and therapeutic delivery of wound healing agents.

### BACKGROUND OF THE INVENTION

The present invention is a device and methods of incorporating wound healing agents, comprised of biological compounds of plant and animal origin, into the three dimensional structure of the precision, porous silicone/nylon membrane described in U.S. patent application Ser. No. 12/049,321 and U.S. patent application Ser. No. 12/326,373, to the current inventor. The present invention performs this function without cross-linking agents in a manner that does not compromise the physical properties of the skin substitute, comprised of stretchability, flexibility, conformability, and other features, nor the aesthetics of the skin substitute.

This new skin substitute can be sterilized using E Beam irradiation without changing its aesthetic, biological or physical properties.

Previously, in the above-referenced applications, methods were described for incorporation of porcine type 1 pharmaceutical grade gelatin (collagen peptides) into the three dimensional structure of the precision porous silicone/nylon membrane. Previous patents to this inventor (Biobrane patent) identified collagen peptide as the best agent for initiating acute adherence of a skin substitute to the wound surface. This is a direct result of interacting with fibrin present in the wound.

The present invention provides similar methods for incorporating the following biological compounds; Vitamin E, Vitamin C, Chondroitin 4 Sulfate, Chondroitin 6 Sulfate and Immuno-10 (a highly purified fraction of Aloe Vera) in addition to porcine collagen peptide into the three dimensional structure of the precision porous silicone/nylon membrane of the referenced applications. Each of these compounds impart a unique biological activity that enhances wound healing.

The Immuno-10 material, U.S. Pat. Nos. 6,436,679, 6,271,214 and 6,133,440, is known for its immunostimulating and immunomodulating properties. Chondroitin Sulfate (4 and 6) is known for its antiflammatory properties. Vitamin E and C are known for their antioxidant properties. Each biological compound has excellent stability at room temperature and is not damaged by exposure to E Beam irradiation.

The ability to directly contact the acute wound surface with these six biological components enables wound healing to occur at a faster rate than normal. The desirable attributes of the skin substitute characterized in the referenced patent applications to this inventor are further enhanced by the addition of five active biological compounds. None of these capabilities are taught in the prior art.

### SUMMARY OF THE INVENTION

The present invention is a method of applying a plurality of biological substances to the surface of a wound by means of a specified, proprietary temporary skin substitute wound dressing. The apparatus is the dressing, comprised of a nylon mesh with proprietary three-dimensional shape and a porous silicone membrane bound to the mesh.

In addition to the substance delivery properties of the invention, the nylon weave in the invention serves to implement three important functions in wound healing, namely fluid transfer, clot support, and skin cell growth. The nylon weave possesses a three-dimensional structure, as in FIG. 4 and FIG. 5, that provides physical and structural support for clots as the skin begins to heal. The porosity of the skin substitute is maintained by the weave at the pore holes, permitting transmission of fluids and preventing transmission of small foreign bodies. The three-dimensional structure of the weave also allows new skin cells to grow in a protected environment.

The substance delivery methods revolve around the application of a plurality of biological and other non-biological substances on and into the apparatus's nylon mesh for the purposes of treating surface injuries of the skin.

### BRIEF OVERVIEW OF THE DRAWINGS AND TABLES

FIG. 1. Vacuum popped hole, standard nylon

FIG. 2. Vacuum-popped hole, low adhesion nylon

FIG. 3. Punctured hole

FIG. 4. Top view of hole showing weave

FIG. 5. Cross-section of vacuum-popped hole showing mesh

Table 1. Biologicals per square cm

### DETAILED DESCRIPTION OF THE INVENTION

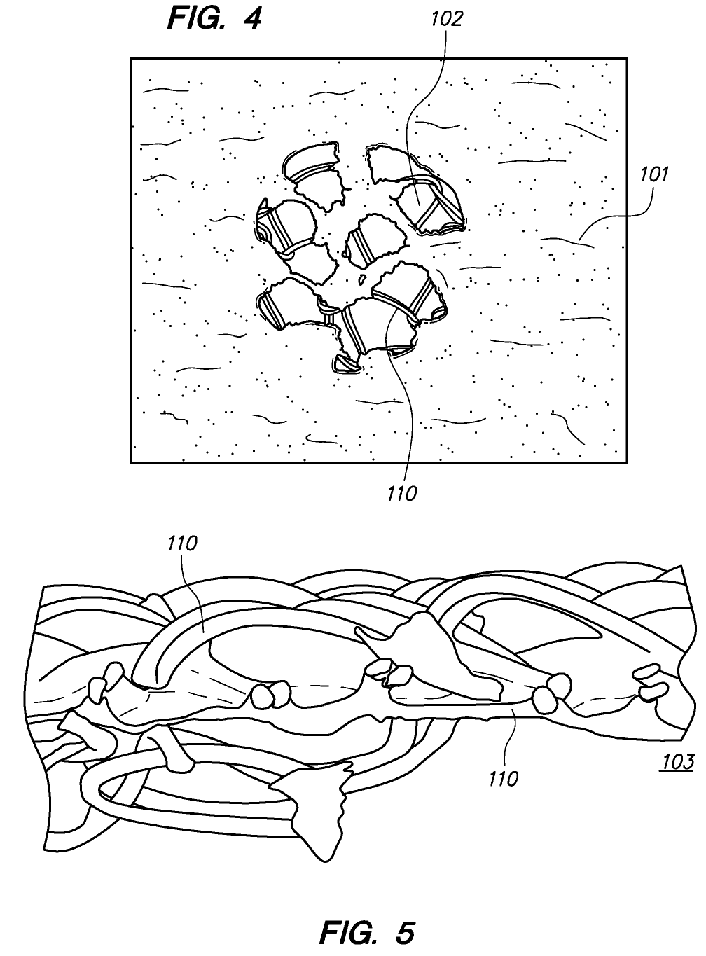

As in FIG. 4 and FIG. 5, the apparatus 100 of the invention is comprised of a silicone membrane 101 with a plurality of holes 102 created in it for porosity. As in FIG. 4 and FIG. 5, physically connected to the membranes 101 by a weave attachment means is a nylon weave 103 comprised of a plurality of interwoven fibers 110 that prevents the passage of gross physical objects larger than 0.001″ through the holes 102.

The range of hole diameters preferred in the present invention is 0.75 mm to 1.05 mm and at holes centered at ¼″-⅓″, providing minimal fluid accumulation beneath the apparatus 100, which will reduce infection complications and maximize wound healing. This is shown in FIGS. 2 and 3.

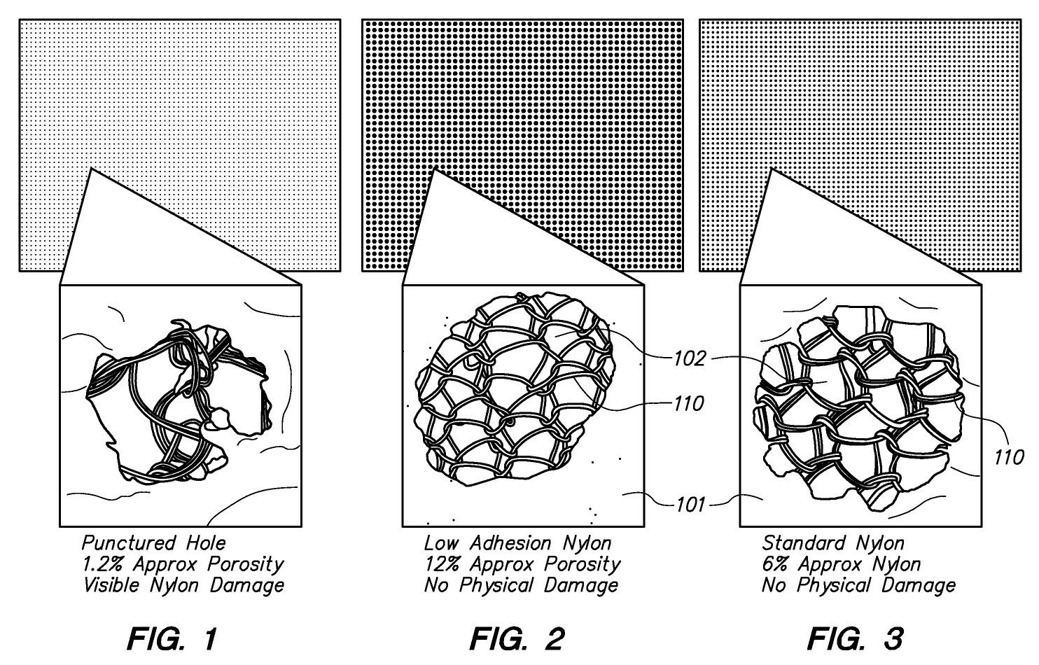

FIG. 1 represents a prior art skin substitute, Biobrane™, which is produced by means of puncturing holes through a similar silicone/nylon weave apparatus. Note that the fibers in the holes are disturbed or destroyed where the fibers 110 in the holes of FIG. 2 and FIG. 3 are relatively undamaged.

The top surface 105 of the apparatus is represented by the silicone membranes 101, the lower surface 106 represented by the nylon weave 103. The two surfaces 105, 106 comprise the two locations for biological and non-biological substances to be applied to the apparatus 100.

The method of use for the apparatus 100 is as a wound dressing or covering. A surface wound of an area determined by the available area of the apparatus 100 can be covered such that fluids can drain from wound through the membrane holes 102, through the weave 13. Similarly, fluids containing drugs or therapeutic agents can pass through the pore holes 102 for the purpose of treating the wound.

As shown in FIG. 4 and FIG. 5, the three-dimensional nature of the weave 103 is designed to provide clot support and cell growth support by means of the structure of the weave 103 itself. The weave 103 is in two qualities, Denier 15/2 and 12/1. Each provides sufficient three-dimensional presence to perform the above functions.

The three-dimensional structure of the weave 103 in combination with use of collagen peptides in the apparatus also provides improved wound adherence for the apparatus; wound adherence is a key property for any skin substitute.

To properly implement the three-dimensional structure of the weave 103 at the pore holes 102, the apparatus 100 is manufactured by means of using vacuum technology instead of puncturing the pore holes 102 into apparatus 100. FIG. 1 shows a typical result at the pore hole as a consequence of puncturing, with disturbed or destroyed weave fibers. FIG. 2 and FIG. 3 show the pore holes 102 produced by the vacuum technology, with an essentially undisturbed weave 103, it's three-dimensional structure intact.

Another method of use of the invention is for therapeutic agents, biological or non-biological, to be applied to the lower surface 106 of the apparatus for direct contact with the wound and surrounding skin.

A third method, and the preferred embodiment of the invention, is the impregnation of the apparatus 100 material itself with substances taken from the list of Vitamin E, Vitamin C, Chondroitin 4 Sulfate, Chondroitin 6 Sulfate and a highly purified fraction of aloe vera (Immuno-10™), and porcine collagen peptide. These substances will be released slowly over time to the covered skin as it comes in contact with the apparatus. Table 1 shows a typical proportion by weight of therapeutic agent applied to the invention.

The Immuno-10™ aloe vera extract is selected for its purity, but the effect provided by aloe vera to the skin is claimed for any fraction of aloe vera used with the invention.

The technology to create the apparatus above and the method listed as the preferred embodiment of this invention, and other methods, are possible and are within the contemplation of this patent.

TABLE 1
Biologicals per Square Centimeter

| | | |
|---|---|---|
| Biologicals | | |
| Gelatin | 2.50000 g | 20.62% |
| Aloe | 7.50000 g | 61.86% |
| Chondroitin Sul. | 0.12500 g | 1.03% |
| Vitamin E | 1.00000 g | 8.25% |
| Vitamin C | 1.00000 g | 8.25% |
| Total Biologicals | 12.12500 g | |
| Pre-Biological wt. | 2.56000 g | 85.62% |
| Post-Biological wt. | 2.99000 g | 100.00% |
| Qnty per 15.24 × 30.48 | 0.43000 g | 14.38% |
| Qnty of Material/cm = | 0.00093 g | |
| Biologicals | 0.00093 g | |
| Qnty per cm² | | |
| Gellatin (g) | 0.00019 g | |
| Aloe (g) | 0.00057 g | |
| Chondroitin Sul. (g) | 0.00001 g | |
| Vitamin E (g) | 0.00008 g | |
| Vitamin C (g) | 0.00008 g | |
| Total Biologicals (g) | 0.00093 g | |

What is claimed is:

1. an artificial skin substitute comprised of a silicone membrane and a nylon weave fabric, the silicone membrane with a plurality of pore holes created in the membrane for porosity,
the silicone membrane attached to the nylon weave with a weave attachment means,
the nylon weave dense enough to prevent the passage of gross physical objects larger than 0.001 inch through the nylon weave and through the pore holes,
the artificial skin substitute possessing a top surface and a bottom surface, the top surface comprised of the silicone membrane, the lower surface represented by the nylon weave,
the two surfaces comprising the two locations for biological and nonbiological substances to be applied to the apparatus,
the nylon weave and silicone membrane impregnateable by biological and nonbiological substances.

2. An artificial skin substitute as in claim 1, where the biological and nonbiological substances used to impregnate the apparatus material are taken from the list comprised of Vitamin E, Vitamin C, Chondroitin 4 Sulfate, Chondroitin 6 Sulfate and aloe vera, and porcine collagen peptide.

3. An artificial skin substitute as in claim 2 where the aloe vera is Immuno10™.

4. A method of use for the artificial skin substitute as in claim 1 comprised of the steps of
obtaining a properly-sized expanse of the skin substitute,
selecting one or more substances with which to impregnate the material of the skin substitute,
Impregnating the material of the skin substitute with the appropriate concentration of said substances,
Applying the skin substitute as a wound dressing to injured skin,
maintaining contact by the skin substitute to the wound for the indicated length of time to accomplish a therapeutic purpose.
